(19) United States
(12) Patent Application Publication
(10) Pub. No.: US 2023/0343469 A1
(43) Pub. Date: Oct. 26, 2023

(54) Method for Predicting Outbreaks of Mosquito-Borne Illness

(71) Applicant: Ngai Kiu Gabrielle Wong, Marlow (GB)

(72) Inventor: Ngai Kiu Gabrielle Wong, Marlow (GB)

(21) Appl. No.: 18/214,779

(22) Filed: Jun. 27, 2023

(51) Int. Cl. G16H 50/70; G16H 30/40

(52) U.S. Cl. CPC G16H 50/70; G16H 30/40

(57) ABSTRACT

Disclosed is a method for predicting an outbreak of mosquito borne illness including receiving a historical weather data, receiving a historical mosquito data, receiving a historical satellite image data, the satellite image data having color data, the color data including at least a near-infrared value and a blue value, processing the historical satellite image data to produce a processed satellite image data. The processing includes, subtracting the blue value from the near-infrared value to obtain a first preprocessing value, adding the blue value to the near-infrared value to obtain a second preprocessing value, dividing the first preprocessing value by the second preprocessing value to obtain a processed blue value. The method further includes tagging the processed satellite image data with the historical weather data and the historical mosquito data, creating a model of mosquito habitats from the processed satellite image data, and comparing a sample satellite image data against the model to determine a mosquito score.

### BACKGROUND OF THE INVENTION

### Field of the Invention

The embodiments of the invention relate to methods of predicting mosquito borne illnesses, and more particularly, to a novel method of processing data to predict a prospective increase in mosquito populations. Although embodiments of the invention are suitable for a wide scope of applications, it is particularly suitable for predicting geographic areas for outbreaks in mosquito borne illness so that local residents can take appropriate prophylactic measures.

### Discussion of the Related Art

The related art for predicting outbreaks mosquito borne illness is an informal science and is not presently governed by a uniform set of rules, is susceptible to individual bias and inconsistent weighting of variables, and is slow in communication and dissemination to the relevant populations. In one example of a hypothetical related art method, a local official for a town may note that the rainy season is coming and advise local residents that mosquito populations swell during the rainy season together with concomitant increase in mosquito borne illness.

Many problems exist with the related art methods. For example, interest in mosquito born illness is of particular interest to residents of sub-Saharan Africa. However, small villages and towns in this region may lack the governmental infrastructure to provide even the most basic of informal predictions. Second, even in areas that have such infrastructure, the manner and means of disseminating this information can be imprecise, delayed, and miscommunicated. Third, the basic relationship between rain and an increase in mosquito populations is an imprecise predictor of the timing and severity of an increase in mosquito populations. Thus, a need exists in the art for a method to predict the timing and severity of increases in mosquito populations in a uniform manner, using all available data, and disseminating the information to interested populations.

### SUMMARY OF THE INVENTION

Accordingly, embodiments of the invention are directed to a method for predicting outbreaks of mosquito-borne illness that substantially obviates one or more of the problems due to limitations and disadvantages of the related art.

An object of embodiments of the invention is to provide an AI model to predict mosquito populations.

Another object of embodiments of the invention is to provide a uniform method of processing mosquito data to predict changes in mosquito populations.

Yet another object of embodiments of the invention is to provide a uniform mosquito score to communicate the relative likelihood of a mosquito outbreak.

Still another object of embodiments of the invention is to provide localized data to interested populations.

Additional features and advantages of embodiments of the invention will be set forth in the description which follows, and in part will be apparent from the description, or may be learned by practice of embodiments of the invention. The objectives and other advantages of the embodiments of the invention will be realized and attained by the structure particularly pointed out in the written description and claims hereof as well as the appended drawings.

To achieve these and other advantages and in accordance with the purpose of embodiments of the invention, as embodied and broadly described, a method for predicting outbreaks of mosquito-borne illness includes receiving a historical weather data, receiving a historical mosquito data, receiving a historical satellite image data, the satellite image data having color data, the color data including at least a near-infrared value and a blue value, processing the historical satellite image data to produce a processed satellite image data. The processing includes, subtracting the blue value from the near-infrared value to obtain a first preprocessing value, adding the blue value to the near-infrared value to obtain a second preprocessing value, dividing the first preprocessing value by the second preprocessing value to obtain a processed blue value. The method further includes tagging the processed satellite image data with the historical weather data and the historical mosquito data, creating a model of mosquito habitats from the processed satellite image data, and comparing a sample satellite image data against the model to determine a mosquito score.

In another aspect, method for predicting outbreaks of mosquito-borne illness includes a receiving a historical weather dataset, the historical weather dataset comprising a plurality of temperatures by date and location, receiving a historical mosquito dataset, the historical mosquito dataset comprising a plurality of reported events of mosquito borne illness by date and location, receiving a historical satellite image dataset, the satellite image dataset comprising a plurality of color data by date and location, each of the color data comprising at least a near-infrared value and a blue value for each date and location, processing the historical satellite image dataset to produce a processed satellite image dataset, by, for each of the plurality of color data, subtracting the blue value from the near-infrared value to obtain a first preprocessing value, adding the blue value to the near-infrared value to obtain a second preprocessing value, dividing the first preprocessing value by the second preprocessing value to obtain a processed blue value, storing the processed blue value to the processed satellite image dataset, correlating the processed satellite image dataset with the historical weather dataset and the historical mosquito data, creating a model of mosquito habitats from the processed satellite image data, and comparing a sample satellite image against the model to determine a mosquito score.

It is to be understood that both the foregoing general description and the following detailed description are exemplary and explanatory and are intended to provide further explanation of embodiments of the invention as claimed.

### BRIEF DESCRIPTION OF THE DRAWINGS

The accompanying drawings, which are included to provide a further understanding of embodiments of the invention and are incorporated in and constitute a part of this specification, illustrate embodiments of the invention and together with the description serve to explain the principles of embodiments of the invention.

FIG. 1 is a process flow chart of a method for predicting mosquito borne illness according to an exemplary embodiment of the invention;

FIG. 2 is a process flow chart of a method for processing satellite data according to an exemplary embodiment of the invention; and

FIG. 3 is a diagram of a sample annotation according to an exemplary embodiment of the invention.

### DETAILED DESCRIPTION OF THE PREFERRED EMBODIMENTS

Reference will now be made in detail to the preferred embodiments of the invention, examples of which are illustrated in the accompanying drawings. The invention may, however, be embodied in many different forms and should not be construed as being limited to the embodiments set forth herein; rather, these embodiments are provided so that this disclosure will be thorough and complete, and will fully convey the concept of the invention to those skilled in the art. In the drawings, the thicknesses of layers and regions are exaggerated for clarity. Like reference numerals in the drawings denote like elements.

Embodiments of the invention may be described herein as a series of steps undertaken in a particular order. The specific ordering of steps described herein is for convenience and is not intended to be limiting. Those of skill in the art will appreciate that certain steps are not dependent on other steps and could be performed in different order or even in parallel and such different order is contemplated and within the scope of the invention. By way of nonlimiting example, embodiments of the invention contemplate collection of data from various sources. Such collection steps can be conducted in any order or in parallel and are within the scope of the invention.

Embodiments of the invention contemplate, generally, collecting data from a variety of sources, processing the data, using the processed data to train a model, and then using the model against sample data to predict mosquito activity of the sample. The method is preferably implemented on a computer that is programmed to perform the method steps more particularly described below. The computer can have a processor and a memory containing programmatic instructions. The processor can read and execute the programmatic instructions to perform the method. The computer may have a network interface card that can communicate with remote databases to receive informational inputs to the method.

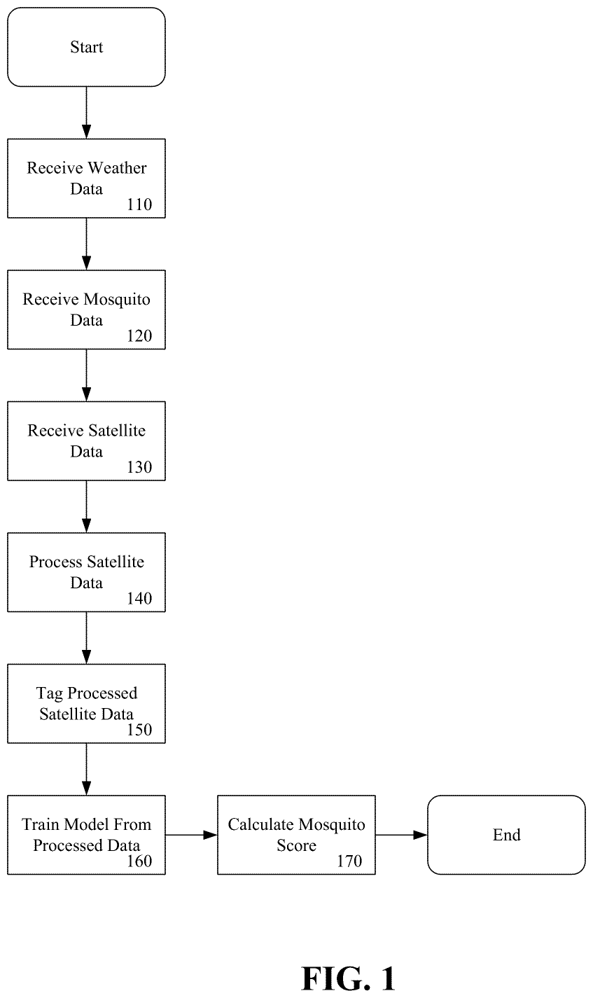

FIG. 1 is a process flow chart of a method for predicting mosquito borne illness according to an exemplary embodiment of the invention. As shown in FIG. 1, a method for predicting mosquito borne illness includes the steps of receiving weather data 110, receiving mosquito data 120, receiving satellite data 130, processing satellite data 140, tagging processed satellite data 150, training a model from processed data 160, and calculating a mosquito score 170.

The process can start at receive weather data step 110. In the receive weather data step 110, weather data can be received from a variety of public sources for an area of interest. For example, weather data for Nigeria is publicly available through the Climate Data Online (CDO) initiative by the National Oceanic and Atmospheric Administration (NOAA). The weather data can include historical weather data over time. For example, the weather data can include daily and location-specific information, including high and low temperatures, high and low humidity, amounts of precipitation, sunrise and sunset times, hours of daylight, average barometric pressure, and average wind speed. Weather data can also include visibility conditions, prevailing weather circumstances, soil moisture, and temperature measurements. The location of the weather data can be a series of latitude and longitude coordinates defining a bounding box for the weather data. The location of the weather data can be a single set of latitude and longitude coordinates where the corresponding data was measured.

In the receive mosquito data step 120, data relating to past occurrences of mosquito activity can be received from a variety of public sources. For example, the authors Kyalo, D., Amratia, P., Mundia, C. W., Mbogo, C. M., Coetzee, M., & Snow, R. W., maintain a geo-coded inventory of anophelines in the Afrotropical Region south of the Sahara from 1898-2016, as documented in their 2017 publication in Wellcome Open Research, which is publicly available and can be accessed at https://doi.org/10.12688/wellcomeopenres.12187.1. The mosquito data can include, for example, measurements of populations of various species of mosquitos at a particular location. Because mosquitos carry various communicable diseases, an increase in the population can indicate a concomitant increase in mosquito borne illness. Additionally, certain species of mosquitos are known to carry certain viruses. For example, the Anopheles mosquito commonly carries malaria and the Aedes mosquito commonly carries zika. Thus, it can be inferred that where there was a past increase in the mosquito population of Anopheles mosquitos that occurrences of malaria similarly increased in the same area at about the same time. The location of the mosquito data can be latitude and longitude coordinates where the corresponding data was measured.

In step 130, satellite data can be received from a variety of public sources. For example, Google Earth Engine maintains a publicly accessible database of historical earth image data. The satellite image data can include date information, location information, and image information. The date information can be the day and time the data was measured. The location information can be latitude and longitude information of where the data was measured. The image information can be the TIFFS or other common image format. The image information can include red, green, blue, (RGB) values and near-infrared (NIR) color values.

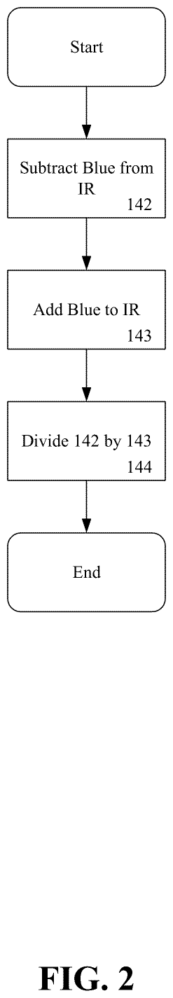

In step 140, the satellite data can be preprocessed for improved performed with popular analytical models. In preferred embodiments of the invention the blue color value (B) of a satellite image data can be preprocessed to alter the blue color value for better compatibility and increased detection by an analytical mode. In one example, the satellite data of step 130 can be preprocessed to remove blue color values. In another example, the blue color value can be offset in accordance with a simple formula such as B=B+1 or B=B+20. As described in greater detail in conjunction with FIG. 2, in preferred embodiments of the invention the blue color value can be subtracted from the NIR value and divided by the NIR value plus the blue color value. Formulaically, this can be represented by B=(NIR−B)/(NIR+B).

With reference to FIG. 2, a detailed process flow chart for preprocessing satellite data is disclosed. Satellite data can include image data for a particular date and location. The image data can include a variety of color values including red, green, and blue (RGB) and near infrared (NIR). In preferred embodiments of the invention the blue value is preprocessed in accordance with the method described in conjunction with FIG. 2 which the inventor has found to be optimal for training a model and detecting mosquito habitats.

In step 142, the blue value is subtracted from the NIR value to calculate a first intermediate value. In step 143, blue value is added to the NIR value to calculate a second intermediate value. In step 144, the first intermediate value from step 142 is divided by the second intermediate value from step 143. The result of step 144 can be the preprocessed blue value that is stored with the corresponding image data and used to build the model described here.

Although the example of FIG. 2 has been described in the context of processing a single image according to the formula B=(NIR−B)/(NIR+B), those of skill in the art will appreciate that the preprocessing is preferably applied to all images of the satellite data thus preprocessing all of the satellite data for use in building the model as will be described in greater detail in conjunction with process step 160 of FIG. 1.

Referring back to FIG. 1, in step 150, the processed satellite data of step 140 can be tagged or correlated with weather data and mosquito data. The respective data can be correlated by date and geographic location. For example, a satellite data for a particular day and location can be correlated with the weather data for the same day and location. Similarly, the satellite data for a particular day and location can be correlated with the mosquito data for the same day and location.

Due to the differing data sources maintained by different entities, it is possible that date data and location data from the respective satellite, weather, and mosquito data do not evenly align. For example, the geographic region for a particular satellite data may be larger than the geographic region for a particular weather data, or vice versa. In such instances, additional preprocessing may be necessary. For example, location data for satellite data may be expressed as a bounding box for the corresponding image data while location data for weather data may be a single point. In such instances, the satellite image data may be tagged or correlated with weather data if the point location of the weather data falls within a bounding box of the location information of the satellite data. In the event two weather data points fall within a single bounding box for satellite data, the two data points may be averaged.

In another example, satellite data may not be available for every day while weather data is available for every day. In one embodiment of the invention, only weather data taken on a day that has a corresponding satellite data for the same day is tagged while other weather data is removed from consideration.

Mosquito data, akin to weather data, is typically collected at a single point and then related to the corresponding satellite data. This correlation or tagging procedure can be similar to the association of weather data with satellite data. Where there is an inconsistency in the alignment of the days and locations between mosquito, weather, and satellite data, only data with corresponding satellite information can be considered. Any data not suitable for correlation with satellite data can be excluded.

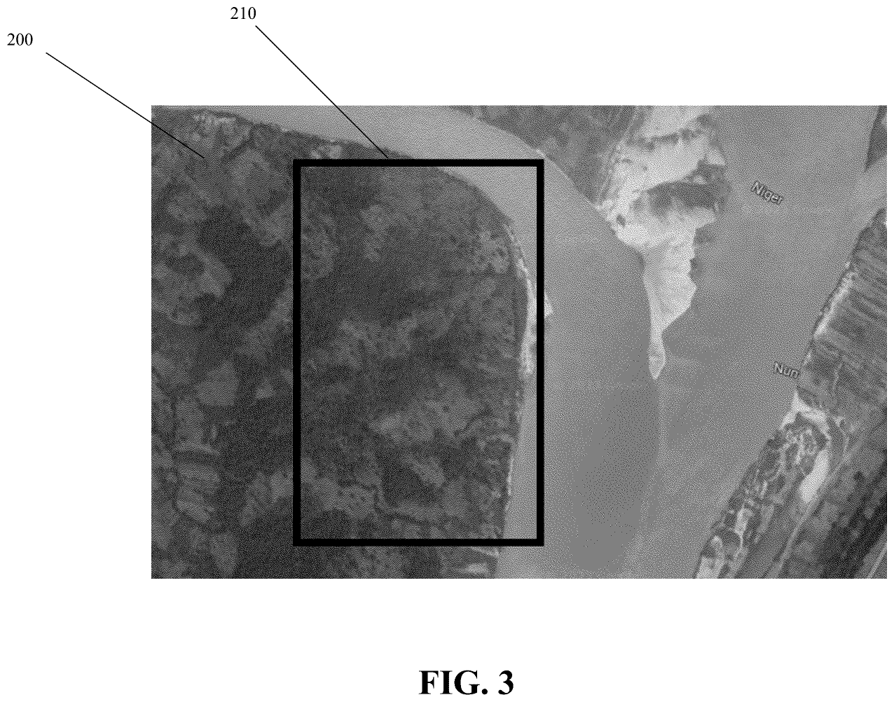

As described in greater detail in conjunction with FIG. 3, the mosquito data can be correlated with satellite data through the derivation of a geographical bounding box representing the extent of mosquito activity in a particular area on a given day. The geographical bounding box can be created in two primary ways—manually or programmatically. In the manual process, the location information indicating mosquito activity can be reviewed by a human that can manually draw a bounding box on the corresponding satellite image data to denote the area of mosquito activity. Alternatively, in a programmatically-driven process, a bounding box can be calculated based on the location information for mosquito activity. An automated process can reduce human error and enhance the efficiency of the data analysis process.

The bounding box, representing a mosquito activity zone, can then be correlated with the satellite data. This process links the mosquito data with corresponding satellite data for a specific day and geographical area, allowing for a comprehensive analysis of mosquito outbreaks based on various environmental factors.

With reference to FIG. 3, an illustration of the manual drawing of a mosquito activity bounding box is described. As shown in FIG. 3, image 200 may be a satellite image of the satellite data. In the exemplary embodiment of FIG. 3, a portion of the Niger river and surrounding land is shown in image 200. The image can be associated with a specific date such as May 20, 2023 at 14:00 hrs. A person manually reviewing mosquito data may determine that a data point of the mosquito data is applicable to a particular geographic area within the area depicted in image 200. The person may use a software tool to draw a bounding box 210 representing the area indicated by the mosquito data. The software tool can translate the drawn bounding box 210 to geographic coordinates within the image 200 to effectively tag or correlate the mosquito data with the satellite data.

Referring back to FIG. 1, in step 160, a model can be trained from the tagged, processed data of step 150. There are numerous machine learning and AI data models readily available and known to those of skill in the art. Examples of such models include, for example, Faster R-CNN, DETR (DEtection TRansformer), and YOLO. The inventor tested multiple such models and determined that YOLOv4 object detection data model provided superior accuracy.

YOLOv4 logically consists of a convolutional neural network backbone, a neck, and a head. The backbone is the data model created by YOLOv4 from processing the sample data (e.g., historical satellite/weather/mosquito data). This backbone serves as the data source used by YOLOv4 to predict attributes of a sample data (e.g., whether an unknown data input is likely to indicate mosquito activity). The neck combines feature layers of the backbone relevant to a particular sample data. The head detects, based on the information passed from the backbone through the neck, attributes of the sample data.

The inventor's experimentation with Faster R-CNN and DETR data models identified prospective benefits, these models ultimately provided less accurate results than YOLOv4 in this specific application. Faster R-CNN uses a region proposal network for bounding box prediction and DETR employs a transformer architecture to predict object presence. These approaches, though effective in many scenarios, were less suited to the specific requirements and data characteristics of predicting mosquito-borne disease outbreaks in this instance.

In step 170, a sample data can be analyzed by YOLOv4 against the model created in step 160 to produce a mosquito score. The mosquito score can indicate the relative likelihood that the sample data is likely to have a high mosquito population. The sample data can be, for example, a satellite image of a particular geographic area tagged with corresponding weather data for the geographic area. The mosquito activity for the geographic area can be initially unknown. Blue color data for the satellite image can be preprocessed using the same method applied to preprocess the historical satellite data as described in step 140. For example, where the historical satellite data was preprocessed using the formula B=(NIR−B)/(NIR+B), the same formula can be applied to preprocess the sample data. YOLOv4 can then process the preprocessed data against the data model to produce a mosquito score.

The mosquito score can represent the likelihood of mosquito activity in the sample image. The mosquito score is preferably a number between zero and one where zero represents lowest likelihood of mosquito activity and one represents highest likelihood of mosquito activity. Those of skill in the art however, will realize that the score can be represented in many ways to convey the same information and the invention is not limited strictly to mosquito scores as set forth herein.

In practice, the model identifies attributes of known mosquito habitats in images and can identify prospective mosquito habitats in unknown images. The model can be more accurate than human analysis in identifying attributes of mosquito habitats, as embodied in a vast database of correlated satellite imagery, weather data, and mosquito data. The inventor has experimentally determined that a model created from unaltered satellite, weather, and mosquito data does not optimally detect mosquito data. Instead, the inventor has determined that adjustments to the blue value of image data produce better detection accuracy than unaltered data and specifically, that altering blue values in accordance with the formula B=(NIR−B)/(NIR+B) can produce an optimized model that yields high mosquito detection rates.

In commercial embodiments of the invention, the backbone model can be created using historical data. The model can be periodically updated as new mosquito data becomes available from locations of known mosquito outbreaks. In this way, the data model can become more robust and benefit from a broader dataset from which to identify characteristics of mosquito habits. In other commercial embodiments, live or delayed satellite data can be combined with live or delayed weather data and compared against the data model to obtain a near real-time mosquito score. Mosquito scores can be overlayed on a map and made available through the web or an app thus enabling individuals in small rural areas the ability to easily leverage the benefits of the invention in their daily lives to inform themselves of the current mosquito risk in near real-time.

Similarly, local governments can be informed of near real-time mosquito risks and take appropriate prophylactic measures such as deploying larvicide Bacillus thuringiensis subspecies israelensis (BTI) to targeted stagnant water bodies, engaging in targeted public awareness campaigns, and proactively ordering medicines to treat mosquito borne illness.

It will be apparent to those skilled in the art that various modifications and variations can be made in the method for predicting outbreaks of mosquito-borne illness without departing from the spirit or scope of the invention. Thus, it is intended that embodiments of the invention cover the modifications and variations of this invention provided they come within the scope of the appended claims and their equivalents.

What is claimed is:

1. A method for predicting outbreaks of mosquito borne illness, the method comprising:
receiving a historical weather data;
receiving a historical mosquito data;
receiving a historical satellite image data, the satellite image data having color data, the color data including at least a near-infrared value and a blue value;
processing the historical satellite image data to produce a processed satellite image data, the processing comprising:
subtracting the blue value from the near-infrared value to obtain a first preprocessing value;
adding the blue value to the near-infrared value to obtain a second preprocessing value;
dividing the first preprocessing value by the second preprocessing value to obtain a processed blue value;
tagging the processed satellite image data with the historical weather data and the historical mosquito data;
creating a model of mosquito habitats from the processed satellite image data; and
comparing a sample satellite image data against the model to determine a mosquito score.

2. The process of claim 1 wherein the tagging of the processed satellite image data with the historical weather data and the historical mosquito data is in accordance with a latitude value and a longitude value in each of the satellite image data, historical weather data, and historical mosquito data.

3. The process of claim 1 wherein the mosquito score indicates a degree of similarity between the sample satellite image data and the model.

4. The process of claim 1 further comprising:
processing the sample satellite image data by subtracting a sample blue value from a sample near-infrared value to obtain a first sample preprocessing value, adding the sample blue value to the sample near-infrared value to obtain a second sample preprocessing value, and dividing the first sample preprocessing value by the second sample preprocessing value to obtain a sample processed blue value.

5. The process of claim 1 wherein the sample satellite image data comprises a sample latitude and a sample longitude.

6. The process of claim 1 wherein the historical weather data comprises a temperature data and a humidity data.

7. The process of claim 1 wherein the historical mosquito data comprises at least one location of a historical outbreak of mosquitos.

8. The process of claim 7 wherein the creating the model includes annotating an image of the historical satellite image dataset with a bounding box representing the at least one location of the historical outbreak of mosquitos.

9. A method for predicting outbreaks of mosquito borne illness, the method comprising:
receiving a historical weather dataset, the historical weather dataset comprising a plurality of temperatures by date and location;
receiving a historical mosquito dataset, the historical mosquito dataset comprising a plurality of reported events of mosquito borne illness by date and location;
receiving a historical satellite image dataset, the satellite image dataset comprising a plurality of color data by date and location, each of the color data comprising at least a near-infrared value and a blue value for each date and location;
processing the historical satellite image dataset to produce a processed satellite image dataset, by, for each of the plurality of color data:
subtracting the blue value from the near-infrared value to obtain a first preprocessing value;
adding the blue value to the near-infrared value to obtain a second preprocessing value;
dividing the first preprocessing value by the second preprocessing value to obtain a processed blue value;
storing the processed blue value to the processed satellite image dataset;
correlating the processed satellite image dataset with the historical weather dataset and the historical mosquito data;
creating a model of mosquito habitats from the processed satellite image data; and
comparing a sample satellite image against the model to determine a mosquito score.

10. The process of claim 8 wherein the location of each of the color data of the historical satellite image dataset is a latitude and a longitude.

11. The process of claim 8 wherein the correlating of the processed satellite image dataset with the historical weather dataset and the historical mosquito dataset is in accordance with a latitude value and a longitude value in each of the satellite image dataset, historical weather dataset, and historical mosquito dataset.

12. The process of claim 8 wherein the mosquito score indicates a degree of similarity between the sample satellite image data and the model.

13. The process of claim 8 further comprising:
processing the sample satellite image dataset by subtracting a sample blue value from a sample near-infrared value to obtain a first sample preprocessing value, adding the sample blue value to the sample near-infrared value to obtain a second sample preprocessing value, and dividing the first sample preprocessing value by the second sample preprocessing value to obtain a sample processed blue value.

14. The process of claim 8 wherein the sample satellite image data comprises a sample latitude and a sample longitude.

15. The process of claim 8 wherein the historical weather dataset further includes, by date and location, a precipitation value, an hours of sunshine value, and a frequency of rain value.

16. The process of claim 8 wherein the creating the model includes annotating an image of the historical satellite image dataset with a bounding box representing one of the plurality of reported events of mosquito borne illness.

17. The process of claim 8 wherein the mosquito score is a value between 0 and 1 where 0 indicating lowest probability of mosquito activity and 1 indicating highest probability of mosquito activity.
